(19) United States
(12) Patent Application Publication
(10) Pub. No.: US 2008/0208008 A1
(43) Pub. Date: Aug. 28, 2008

(54) Wireless body sensor with small size background

(75) Inventor: James B. Turner, Monroe, WA (US)

Correspondence Address: MERCHANT & GOULD (MICROSOFT), P.O. BOX 2903, MINNEAPOLIS, MN, 55402-0903, US

(73) Assignee: Microsoft Corporation, Redmond, WA (US)

(21) Appl. No.: 11/712,766

(22) Filed: Feb. 28, 2007

(51) Int. Cl. A61B 5/04

(52) U.S. Cl. 600/300

(57) ABSTRACT

A wireless body sensor device includes a number of small probes that are arranged about a wireless electronics system. The wireless electronics system can process bio-potential signals from the probes and communicate bio-potential data to another device such as a wireless telephone, wrist watch, personal data assistant (PDA), laptop computer or any other appropriate device. By forming the probes within a confined short range of the wireless electronics system, interference and noise is greatly reduced. The wireless electronics system includes signal amplifiers that increase the signal levels associated with the bio-potential probes so that the signals can be converted to bio-potential data through an analog-to-digital conversion (ADC) process. Once the bio-potential data is in digital form, the data can be processed by a digital signal processor (DSP), encoded into a signal transmission, and communicated to another device with a radio system and its corresponding antenna.

### BACKGROUND

Body sensor systems are currently used for in health diagnostics and in health maintenance applications. Example applications for body sensor systems include: monitoring sports performance, diagnosing metabolic disorders, identifying sleep disorders, as well as monitoring specific body systems cardiac and neurological systems.

Some example body sensor systems are used for processing simple physiological data, while other body sensor systems are used for processing bio-potential data. Simple physiological data includes measurements for physiological characteristics such as blood pressure, heart rate, skin temperature, and body temperature, to name a few. On the other hand, bio-potential data is a measurement of differences in potential between two points of interest on the body. Bio-potential data is often associated with measurements such as electro-cardiograms (ECG/EKG), where the electrical voltages for heart activity are monitored, electro-encephalograms (EEG), where electrical voltages for brain activity are monitored.

Each body sensor system is comprised of one or more sensor devices and a signal processing system. Each sensor device is affixed to the body region that is being monitored, where electrical signals that are generated by the sensor devices are collected by the signal processing system. The signal processing system includes capabilities for performing amplification, signal conditioning, filtering, and analog-to-digital conversion, to name a few. Some other signal processing systems also include the capability of providing data logging functions.

In simple sensory systems, the sensor devices and the signal processing system are provided in a wearable form factor. Wearable body sensor systems are available for measuring various physiological data such as: blood pressure, heart rate, body temperature, and many others. Wearable body sensor systems often combine the sensors for measuring the physiological data together with the signal processing system in a single wearable device such as a watch or armband device.

The signal processing system is remotely located from the sensors in complex sensory systems. In one example, the individual sensors span too great a physical distance to be located on a single wearable device. In another example, the sensors are physically located in places where a wearable signal processing device is not practical. In such complex sensory systems, wired sensors are affixed to key locations of the body, and the wires from the various sensors are collectively connected to the remote located signal processor. In some instances the remotely located signal processor can be worn in a hip-pack, while in other instances it is necessary to use a non-wearable signal processing unit such as in a hospital or clinical setting where complex instrumentation is utilized.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure is related to devices, methods and systems for sensing and processing bio-potentials. A wireless body sensor device includes a number of small probes that are arranged about a wireless electronics system. The wireless electronics system can process bio-potential signals from the probes and communicate bio-potential data to another device such as a wireless telephone, wrist watch, personal data assistant (PDA), laptop computer or any other appropriate device. By forming the probes within a confined short range of the wireless electronics system, interference and noise is greatly reduced. An array of the wireless body sensor devices can be mapped into a Cartesian coordinate system for recording a multiplicity of readings that can later be mapped into familiar representations such as an EEG or other bio-potential measurement system that is known to health care workers.

In one example implementation of a wireless body sensor device, bio-potential probes are coupled to the inputs of signal amplifiers. Each signal amplifier increases the signal level associated with the bio-potential probes so that the signals can be converted to bio-potential data through an analog-to-digital conversion (ADC) process. Once the bio-potential data is in digital form, the data can be processed by a digital signal processor (DSP), encoded into a signal transmission, and communicated to another device with a radio system and its corresponding antenna. The radio system and antenna can be arranged for any desired transmission format such as a Bluetooth transmission, a radio frequency (RF) transmission, or an ultra-wideband (UWB) transmission.

In a further example implementation of a wireless body sensor device, a signal shaper can be included in the radio/antenna system so that the waveforms that are coupled to the antenna are pre-distorted to provide a desired result. The pre-distortion can be arranged to compensate for the signal dispersion characteristics of the antenna such that the signal transmitted by the antenna corresponds to a dispersion free Gaussian mono-pulse signal in the time domain.

In another example implementation of a wireless body sensor device, a Micro-electro-mechanical system (MEMS) switch array can be used to couple the bio-potential probes to the sense inputs of signal amplifiers. The operation of the MEMS switch array can optionally be controlled by a control processor such that the inputs of the signal amplifiers are auto-zeroed. The operation of the MEMS switch array can also optionally be controlled by the control processor such that the DC offsets and drift associated with the inputs to the signal amplifiers are removed. Moreover, the operation of the MEMS switch array can also optionally be controlled by the control processor such that the signal amplifiers are chopper stabilized with a desired noise shaping characteristic.

In an example implementation of a system employing a wireless body sensor device, each wireless body sensor device has four bio-potential probes that are arranged about its wireless electronics system. The bio-potentials from each of the probes can be processed into a vector form that maps three distinct spatial directions for each wireless sensor device. The data collected from the collection of wireless body sensor devices can then be processed by the system into other representations such as one of the standard forms familiar to health care workers.

These and other features and advantages will be apparent from reading the following detailed description and reviewing the associated drawings. It is to be understood that both the foregoing general description and the following detailed description are explanatory only and are not restrictive. Among other things, the various embodiments described herein may be embodied as methods, devices, or a combination thereof. Likewise, the various embodiments may take the form of a hardware embodiment, a software embodiment or an embodiment that combines software and hardware aspects. The disclosure herein is, therefore, not to be taken in a limiting sense.

### BRIEF DESCRIPTION OF THE DRAWINGS

FIG. 1 is a diagram illustrating an example application for the wireless body sensor devices of the present disclosure.

FIG. 2 is a diagram illustrating the communication of a wireless body sensor device with various other devices in accordance with the present disclosure.

FIG. 3 is a schematic diagram illustrating an example computing device that is configured for operation according to the present disclosure.

FIGS. 4A and 4B are diagrams illustrating detailed examples of wireless body sensor devices configured according to the present disclosure.

FIG. 5 is a diagram illustrating an ultra-wideband antenna for a wireless body sensor device that is arranged in accordance with the present disclosure.

FIG. 6 is a diagram illustrating signal dispersion and correction in a wireless body sensor device that is arranged in accordance with the present disclosure.

FIG. 7 is a flow diagram illustrating an implementation of a method employed by a wireless body sensor device to process bio-potentials in accordance with the present disclosure.

FIG. 8 is a flow diagram illustrating an implementation of a method employed by a wireless body sensor device for relaying transmissions in accordance with the present disclosure.

FIG. 9 is a flow diagram illustrating an implementation of a method employed by a system for receiving and processing bio-potential data from a wireless body sensor device that is arranged in accordance with the present disclosure.

### DETAILED DESCRIPTION OF IMPLEMENTATIONS

This detailed description describes implementations of a wireless body sensor device and applications in bio-potential measurement systems. Generally, a wireless body sensor device includes one or more bio-potential probes, and a wireless electronics system. The overall size and form factor of the wireless electronics system is on the order of 15 mm by 15 mm, with a thickness of about 5 mm. This form factor is sufficiently small such that a wireless electronics system can be affixed to a very small battery such as a watch battery. Each of the probes is arranged to contact a surface to measure bio-potentials, and to provide bio-potential signals to the wireless electronics system for processing and transmission to other devices such as may be used in health diagnostics applications. The measured bio-potentials may also have applications in sports training situations, as well as entertainment purposes such as in video games.

Briefly stated, a wireless body sensor device includes a number of small probes that are arranged about a wireless electronics system. The wireless electronics system can process bio-potential signals from the probes and communicate bio-potential data to another device such as a wireless telephone, wrist watch, personal data assistant (PDA), laptop computer or any other appropriate device. By forming the probes within a confined short range of the wireless electronics system, interference and noise is greatly reduced. The wireless electronics system includes signal amplifiers that increase the signal levels associated with the bio-potential probes so that the signals can be converted to bio-potential data through an analog-to-digital conversion (ADC) process. Once the bio-potential data is in digital form, the data can be processed by a digital signal processor (DSP), encoded into a signal transmission, and communicated to another device with a radio system and its corresponding antenna.

Existing bio-potential systems include probes that are affixed to the patient (e.g., adhesively applied to the skin surface of the patient) with long wires that connect the probes to a centralized signal processing unit. The systems, methods and devices described herein allow for small sensor nodes to be worn by the patient for extended periods of time. Bio-potential data is wirelessly communicated to a portable processing unit (e.g., a cell phone, a PDA, a wristwatch, etc.) for any number of applications including real-time processing and data logging applications. Logged data can be later transmitted to another device such as a central processing unit for further computer processing, analysis, and/or storage.

EEG and other bio-potential measurement systems have common methods of presentation to allow observers to make decisions concerning the patient's health. The present disclosure recognizes that modern EEG systems with long wires have problems with signal interference and noise from power wiring in buildings. The presently described devices utilize probes with very short wire connections to the electronic signal processing circuitry so that signal interference and noise are greatly reduced.

The present disclosure also recognizes that long wire systems have additional problems in that mechanical shock to the wires causes false signals that appear as electrical impulses. The presently disclosure devices with very small sensors and short lengths of wire connecting the bio-potential probes to the signal processing electronics also avoids the described mechanical shock problem.

Since the distance between the bio-potential probes are very small, conventional methods of measurement points are unavailable. Multiple wireless body sensor devices can be placed on the patient in a plurality of sensor locations so that additional measurements can be collected. The various measurements from the wireless body sensor devices can be mapped from a Cartesian coordinate system back into a more familiar presentation.

### Examples Wireless Body Sensor Application

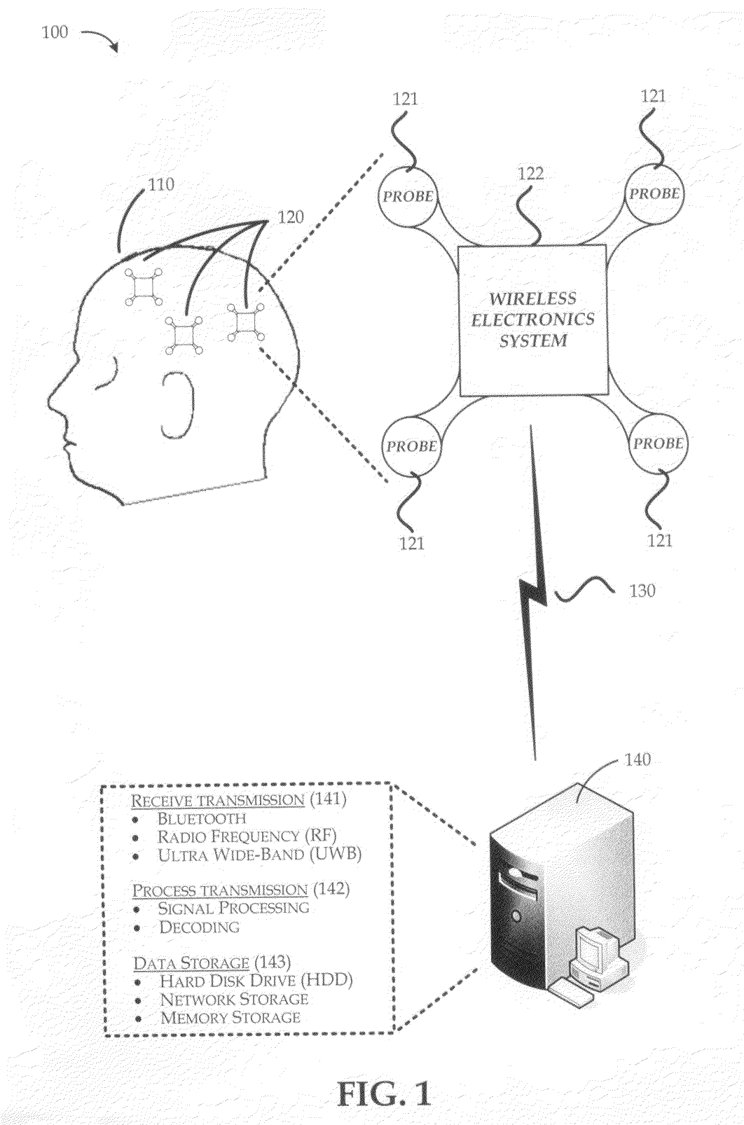

FIG. 1 is a diagram illustrating an example application (100) for the wireless body sensor devices of the present disclosure. As illustrated in the figure, a user (110) of the system has a plurality of wireless body sensor devices (120) affixed to a skin surface for collecting measurements of bio-potentials. Each wireless body sensor device (120) includes two or more bio-potential probes (121) that are coupled to a wireless electronics system (122). The wireless electronics system is arranged to communicate bio-potential data to a computing device (140) via a wireless transmission (130).

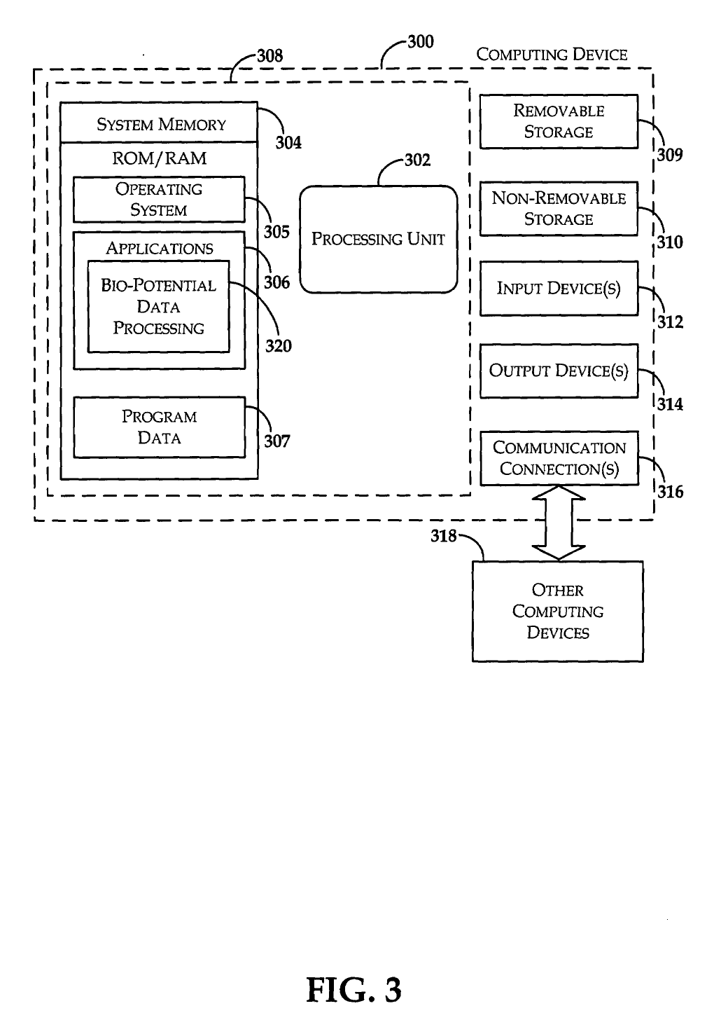

The computing device (140) is arranged to receive the transmission (141), process the transmission (142) and perform any necessary data storage operations (143). The transmission reception (141) is matched to the format of the wireless transmission (130), which can be provided in any appropriate format including, but not limited to a Bluetooth transmission, a radio-frequency transmission, and an ultra-wideband (UWB) transmission. The transmission processing (142) can include signal processing functions such as filtering, amplification, and decoding functions, to name a few. The data storage operations (143) can includes storage to a hard disk drive (HDD), a network storage device, or to a memory device. Additional details about computing devices will be described with reference to FIG. 3.

In the example depicted in FIG. 1, the wireless body sensor devices are depicted as affixed to a user's head such as might be required for an EEG measurement. However, any other skin surface may be applicable for other measurements such as, for example, affixing wireless body sensor devices on the user's chest in the case of EKG measurements.

### Examples Computing Devices

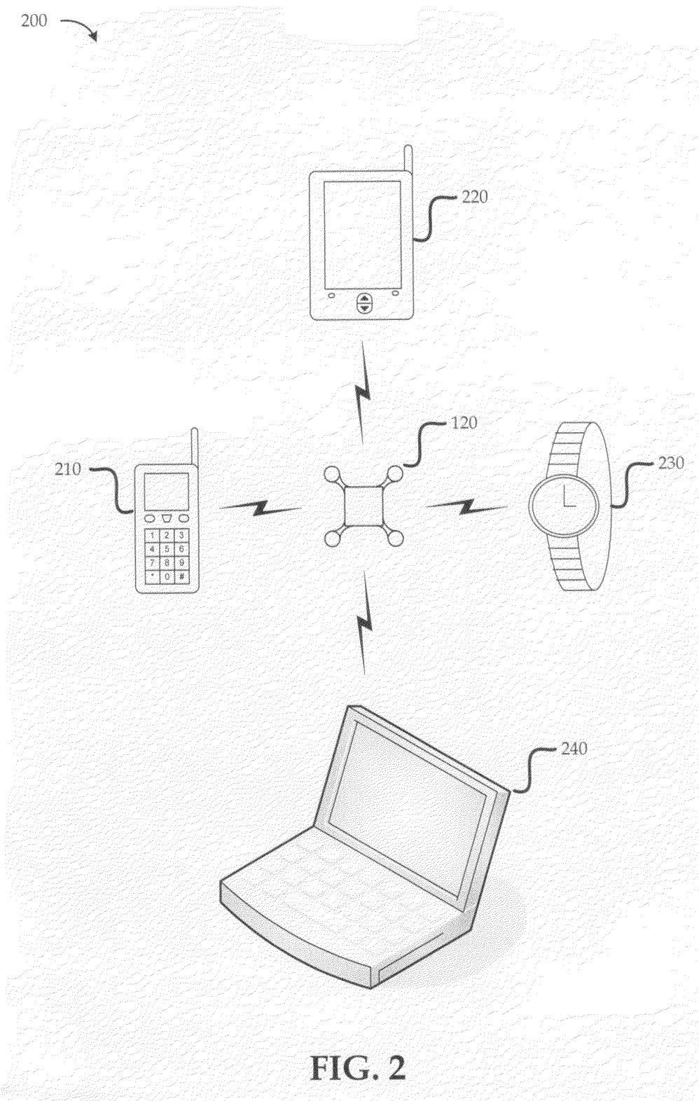

FIG. 2 is a diagram (200) illustrating the communication of a wireless body sensor device with various computing devices in accordance with the present disclosure. As depicted in FIG. 2, example computing devices that are arranged for communicating with the wireless body sensor device include portable devices and non-portable devices. Example portable computing devices include cellular telephones (210), personal data assistants (220), wrist watch devices (230), and personal computers such as a laptop computer (240).

FIG. 3 is a schematic diagram illustrating an example computing device (300) that is configured for operation according to the present disclosure. In a very basic configuration, computing device 300 typically includes at least one processing unit (302) and system memory (304). Depending on the exact configuration and type of computing device, system memory 304 may be volatile (such as RAM), non-volatile (such as ROM, flash memory, etc.) or some combination of the two. System memory 304 typically includes an operating system (305), one or more applications (306), and may include program data (307). Application 306 includes a bio-potential data processing algorithm (320) that is arranged to process data for either real-time processing applications (e.g., monitoring bio-potentials), or for data storage and transfer applications (e.g., data storage, retrieval, encoding, decoding, etc.). In one embodiment, application 306 further includes a user interface for displaying real-time bio-potential data, or for providing alerts in response to bio-potential data. This basic configuration is illustrated in FIG. 3 by those components within dashed line 308.

Computing device 300 may have additional features or functionality. For example, computing device 300 may also include additional data storage devices (removable and/or non-removable) such as, for example, magnetic disks, optical disks, or tape. Such additional storage is illustrated in FIG. 3 by removable storage 309 and non-removable storage 310. Computer storage media may include volatile and nonvolatile, removable and non-removable media implemented in any method or technology for storage of information, such as computer readable instructions, data structures, program modules, or other data. System memory 304, removable storage 309 and non-removable storage 310 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 300. Any such computer storage media may be part of device 300. Computing device 300 may also have input device(s) 312 such as keyboard, keypad, mouse, pen, voice input device, touch input device, etc. Output device(s) 314 such as a display, speakers, printer, etc. may also be included.

Computing device 300 also contain communication connections 316 that allow the device to communicate with other computing devices 318, such as over a network. Communication connection 316 is one example of communication media. Communication media may typically be embodied by computer readable instructions, data structures, program modules, or other data in a modulated data signal, such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency (RF), infrared (IR), ultra-wideband (UWB), Bluetooth and other wireless media. The term computer readable media as used herein includes both storage media and communication media.

Computing device 300 can be implemented as a portion of a small-form factor portable electronic device such as a cell phone, a personal data assistant (PDA), a personal media player device, a wireless web-watch device, or a hybrid devices that include any of the above functions. Computing device 300 can also be implemented as a personal computer including both laptop computer and non-laptop computer configurations.

### Examples Wireless Body Sensor Device

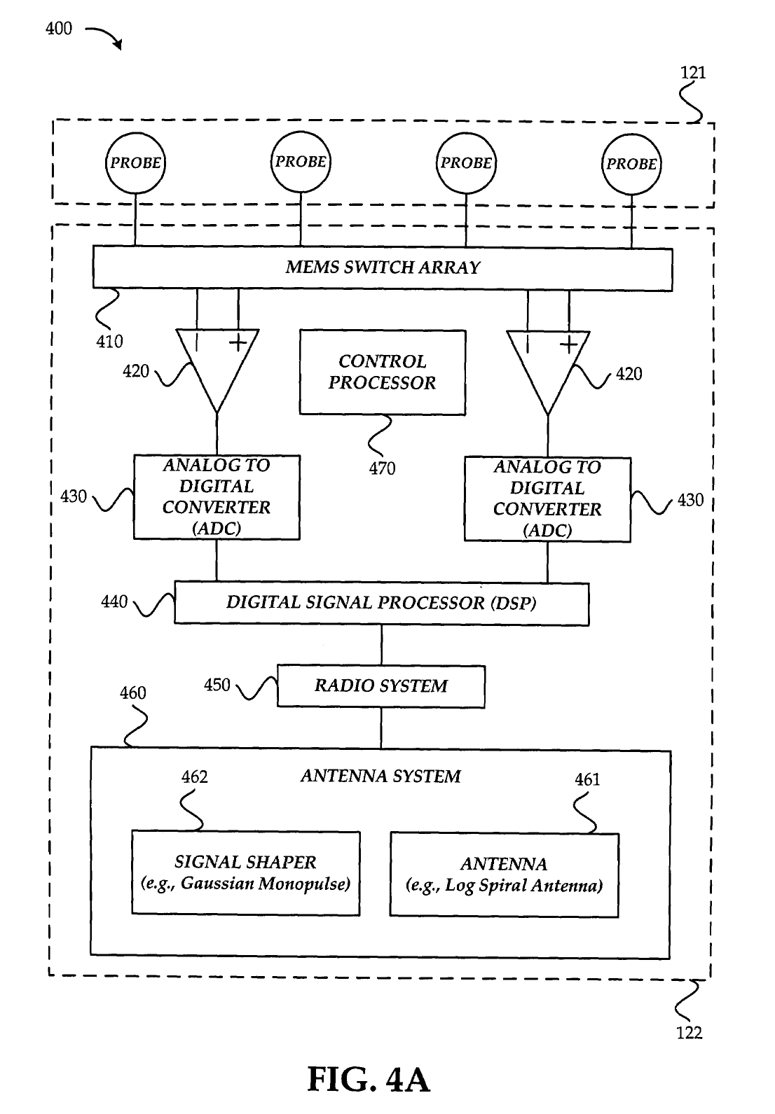

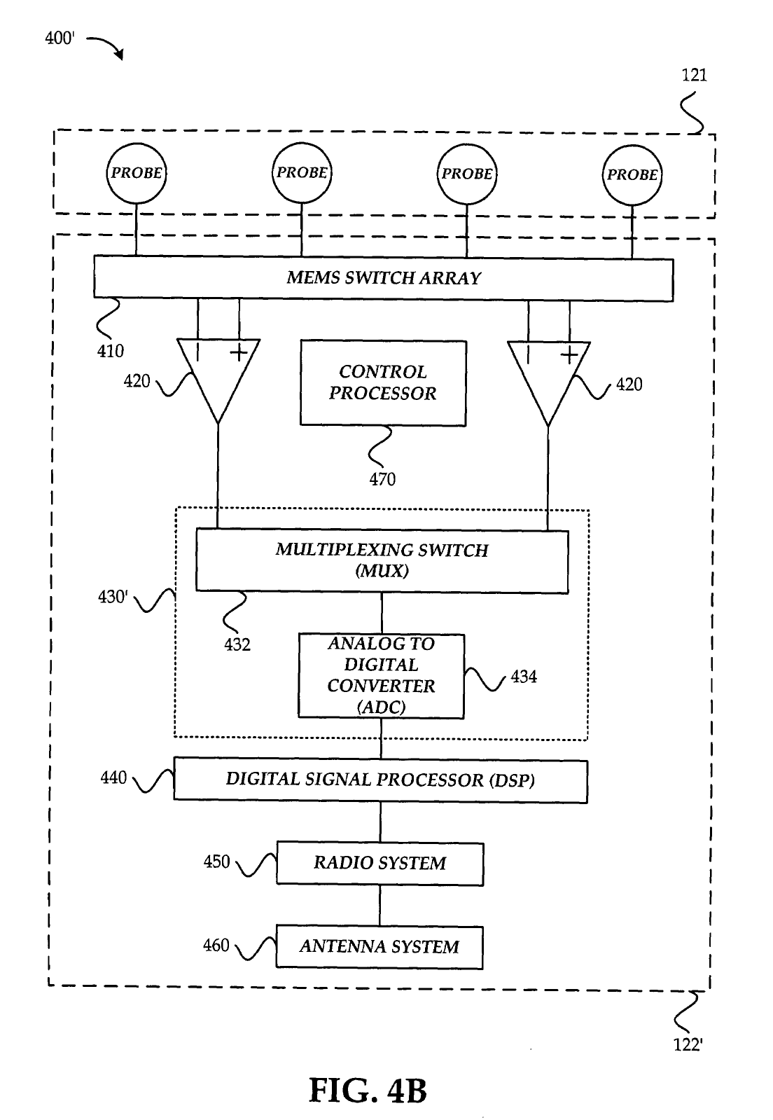

FIGS. 4A and 4B are diagrams illustrating detailed examples of wireless body sensor devices (400 and 400′) configured according to the present disclosure.

The wireless body sensor device (400) of FIG. 4A includes bio-potential probes (121) that are coupled to a wireless electronics system (122) as described previously. The bio-potential probes (121) are coupled to the inputs of signal amplifiers (420) in the wireless electronic system (122) through an optional MEMS switch array (410). Each signal amplifier (420) increases the signal level associated with the bio-potential probes so that the signals can be converted to bio-potential data through via an ADC circuit which is represented in FIG. 4A as two separate ADCs (430). Once the bio-potential data is in digital form, the data can be processed by a DSP circuit (440), and encoded into any appropriate format for a radio system (450). The radio system (450) operates with an antenna system (460) to transmit signals to another device (e.g., see FIGS. 1 and 2). When utilized in the wireless body sensor device, the MEMS switch array (410) is arranged to selectively switch each of the bio-potential probes to a respective input of a signal amplifiers (420), under control of the control processor (470).

The wireless body sensor device (400′) of FIG. 4B also includes bio-potential probes (121) that are coupled to a wireless electronics system (122′) as described previously. The wireless electronics system (122′) depicted in FIG. 4B is substantially similar to that described for FIG. 4A, with identical labels for identical components. However, in FIG. 4B the two ADCs (430) from FIG. 4A have been replaced with a multiplexer switch or MUX (432) and a single ADC (434). The multiplexer switch (432) is arranged to selectively couple an output from one of the signal amplifiers (420) to the ADC (432) for analog-to-digital conversion.

The radio system (450) and antenna system (460) are arranged to transmit the bio-potential data in any desired transmission format such as a Bluetooth transmission, a radio frequency (RF) transmission, or an ultra-wideband (UWB) transmission, as previously described.

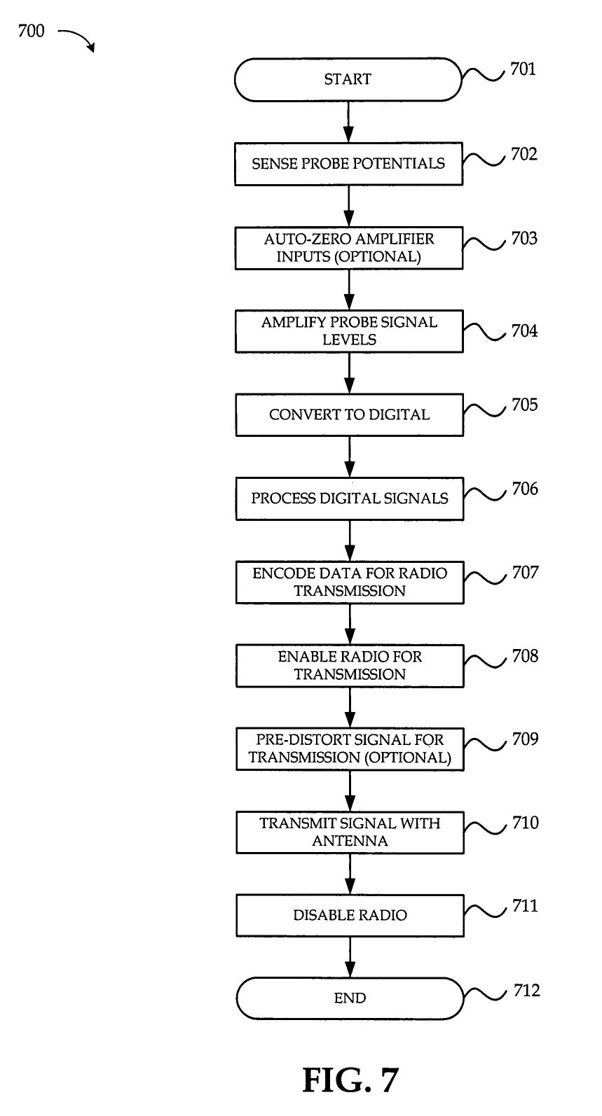

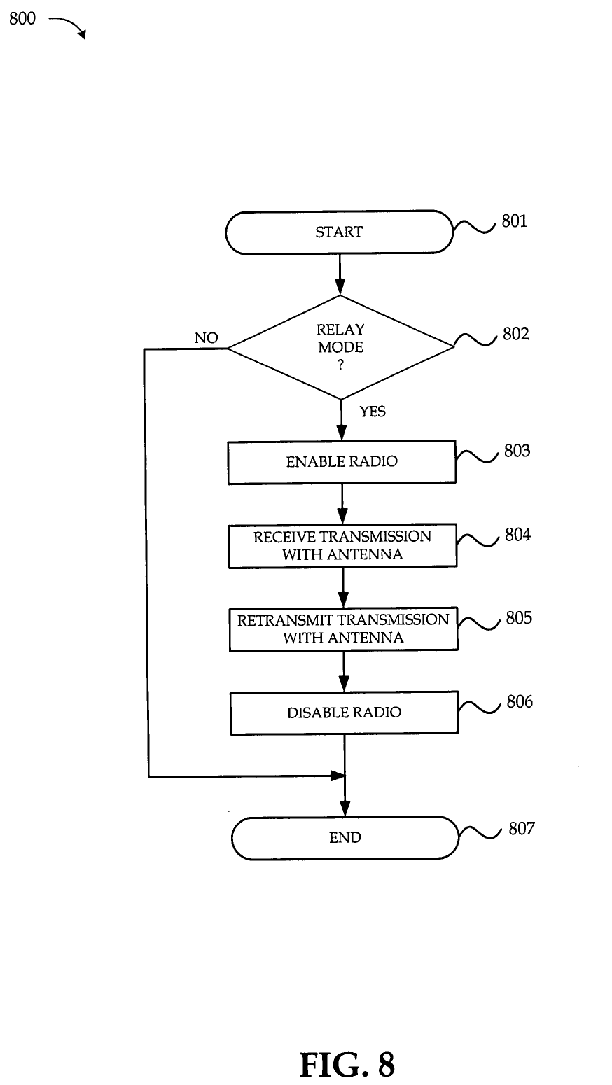

The antenna system includes an antenna (461) and an optional signal shaper (462). The signal shaper (462) can be included in the antenna system (462) so that the waveforms that are coupled to the antenna are pre-distorted to provide a desired result. The pre-distortion can be arranged to compensate for the signal dispersion characteristics of the antenna such as by shaping the signals similar to a Gaussian mono-pulse in the time domain, as will be described later with respect to FIGS. 7 and 8.

The wireless body sensor device (400) is designed to satisfy various constraints such as using small surface area, closely spaced bio-potential probes. Since the bio-potential probes (121) are located close to one anther, the sensed voltages will be very small. The low voltage from the bio-potential probes drives the input characteristics of the signal amplifiers (420). It is thus required that the signal amplifiers exhibit characteristics of very low noise and high common mode rejection.

Lighting and power wiring in conventional buildings tend to cause large magnetic fields with a nominal frequency around 50 Hz or 60 Hz. Conventional bio-potential measurement systems use probe wires that can exceed 12 inches in length, which cause large voltages at the power frequencies to couple into the signal processing circuits of the nodes in conventional systems. Since the bio-potential probes of the present disclosure are located in extremely close proximity to the signal processing electronics (e.g., signal amplifiers 420, etc.), there is nominally no coupling of signals from the power frequencies into the signal processing electronics.

Bio-potential probes can have signals that exhibit large DC offsets that often change dynamically during use. The DC offsets can be quiet large compared to the signals being measured. One approach to minimize the effects of DC offset is to use a coupling circuit between the bio-potential probes (121) and the signal amplifiers (420) that block voltages from DC up to about 0.5 Hz. The MEMS switch array (410) can be arranged between the bio-potential probes (121) and the signal amplifiers (420) to periodically switch the polarity of the bio-potential probes so that the offset can be eliminated. The system software can then be configured to recognize that sampled data should be modified by a give DC value. Such methods are sometimes called auto-zero circuits or offset nulling circuits.

A method of removing the DC and DC drift due to the signal amplifiers (420) is to periodically change the input configuration of the MEMS switch array (410) at a rate higher than the frequency where the major errors of the signal amplifiers (420) are present. This periodic switching has the effect of modulating the bio-potential to a double side band signal centered at the switching frequency. The DC input levels and DC drift of the signal amplifiers (420) can be filtered out before the sampling occurs in the ADC circuits (430). Advantageously, the MEMS switching array (410) does not have the same noise characteristics that would be present in semi-conductor switching.

The wireless body sensor device is constrained in size and weight such that it is well suited for implementation as an integrated circuit. Integrated circuits have multiple functions and small packaging, which will allow the total size for each wireless body sensor device to be small (less than 2 cm, on the order or 1 cm per device, with a 15 mm by 15 mm electronics portion). The size and weight of the overall device is also constrained by the size and weight associated with the battery. Integrated circuits used for the wireless electronics system (122) should be very low power as well as high performance. The control processor (470) can also be arranged to disable the radio portion of the electronics until needed so that battery power is conserved. The DSP (440) can also be configured to allow extraction of certain waveform features and only transmit the feature statistics rather than the bio-potential data (i.e., the sampled version of the amplified bio-potentials from the probes) to further reduce power consumption.

Ideally, the battery for the wireless electronics system is rechargeable. Although higher energy density is available from batteries that are non-rechargeable, the various power reduction methods described herein should reduce energy consumption sufficient such that batteries would not need to be recharged more than once a day.

For reliable radio links each wireless body sensor device (120) can be configured to act as a relay that receives and resends data from other nodes. Retransmission requirements for resending between nodes can be coordinated by the processing unit (e.g., wrist watch, cell phone, personal computer, etc.).

In an example implementation illustrated in FIGS. 1, 2 and 4, the wireless body sensor device has four bio-potential probes that are arranged about its wireless electronics system. The bio-potentials from each of the probes can be processed into a vector form that maps three distinct spatial directions for each wireless sensor device. The data collected from the collection of wireless body sensor devices can then be processed by the system into other representations such as one of the standard forms familiar to health care workers.

### Examples Antenna Design

The FCC has recently allowed new methods of wireless transmission in an unlicensed deployment referred to as Ultra-Wideband or UWB. Using an UWB radio can reduce total power consumption of the wireless body sensor device. However, a compact UWB antenna can have time dispersion effects when used with an impulse radio. Such time dispersion makes the correlation at the receiver less accurate. To overcome the problem, the antenna system (460) is arranged to change the pulses at the antenna (461) input in a manner so that after the signal is received, the pulse no longer exhibits dispersion.

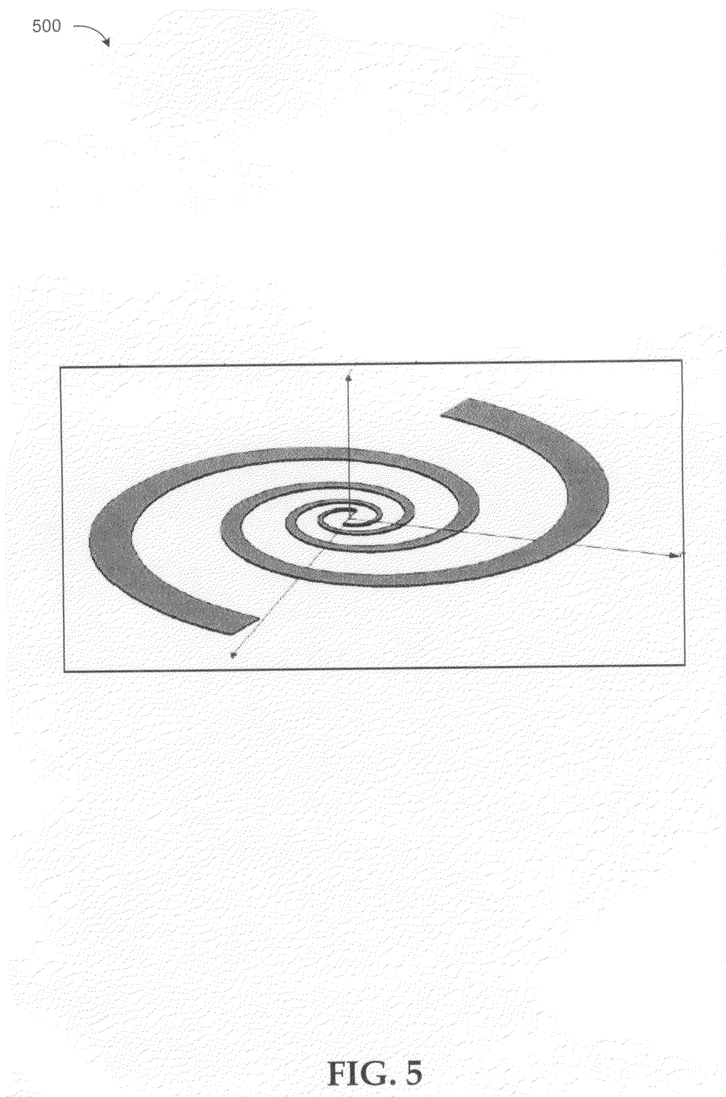

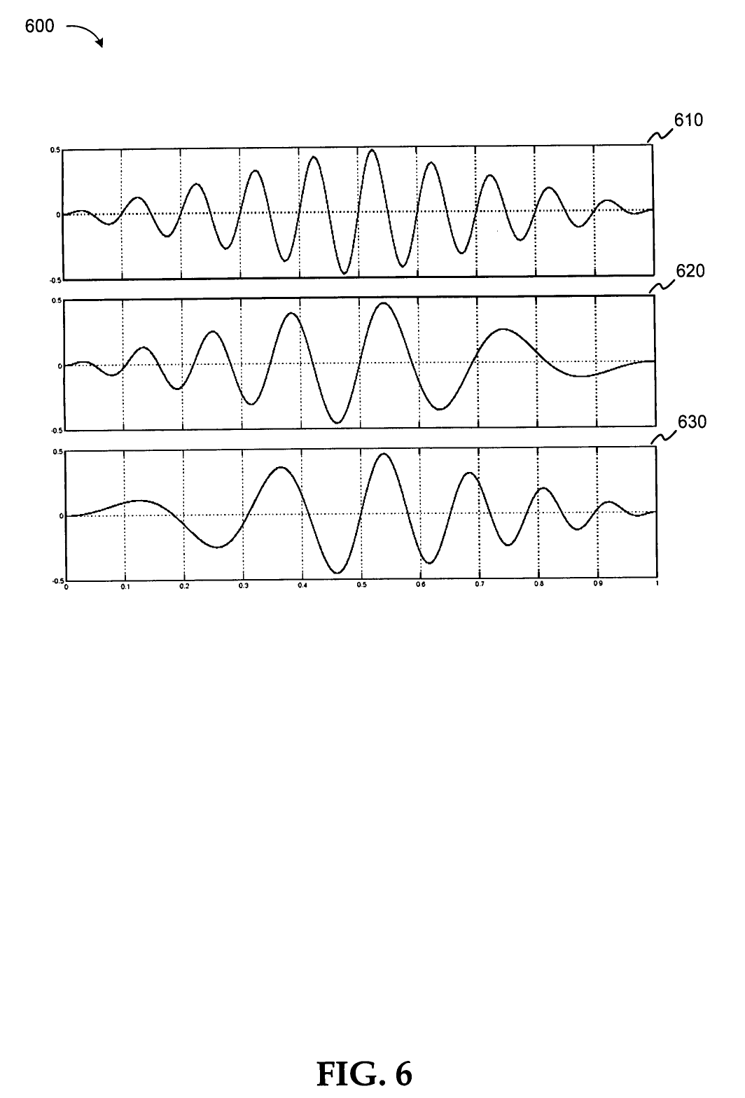

FIG. 5 is a diagram illustrating an ultra-wideband antenna (500) for a wireless body sensor device that is arranged in accordance with the present disclosure. The antenna (500) is designed as a spiral antenna such as a log spiral. FIG. 6 is a diagram (600) illustrating signal dispersion and correction in a wireless body sensor device that uses the UWB antenna of FIG. 5. As illustrated in FIG. 6, the top waveform (610) is a wideband pulse signal without any signal dispersion with a Gaussian shape in the time domain. The middle waveform (620) shows how a waveform is distorted by signal dispersion. The bottom waveform (630) shows a waveform that has been pre-distorted to account for signal dispersion so that after the effects of transmission via the antenna, the resulting waveform will look more like the ideal Gaussian shape depicted as the top waveform (610).

### Examples Process Flows

Once bio-potentials are processed and sampled, the bio-potential data needs to be transmitted to a device that can either process, log or display the results. The bio-potential data can be transmitted directly to a personal computer or to another computing device such as a wristwatch, cell phone or some other mobile computing device as previously described.

FIG. 7 is a flow diagram (700) illustrating an implementation of a method employed by a wireless body sensor device to process and transmit bio-potentials in accordance with the present disclosure. Processing begins at block 701, and proceeds to block 702 where the bio-potential probes sense various potentials. At block 703, the MEMS switch array can optionally provide auto-zero functions to the inputs of the signal amplifiers. Continuing at block 704, the signals from the bio-potential probes are coupled to the signal amplifiers and produce amplified signals. At block 705, the amplified signals are converted into digital signals which are then processed at block 706. When it is time to transmit data to a computing device, the data is encoded for transmission at block 707, and the radio system is enabled for transmission at block 708. At block 709 the signals from the radio system can optionally be pre-distorted before transmission via the antenna at block 710. The radio is disabled at block 711 so that power is conserved. Processing ends at block 712.

FIG. 8 is a flow diagram (800) illustrating an implementation of a method employed by a wireless body sensor device for relaying transmissions in accordance with the present disclosure. Processing begins at block 801, and proceeds to decision block 802 where the wireless body sensor device determines if the relay mode is active. When the relay mode is not active processing terminates at block 807. Otherwise, when the radio is enabled at block 803 when the relay mode is active. Once the radio is enabled, a transmission is received via the antenna at block 804, and the radio system will retransmit the received information at block 805. After the retransmission is completed, the radio is again disabled at block 806 and processing terminates at block 807.

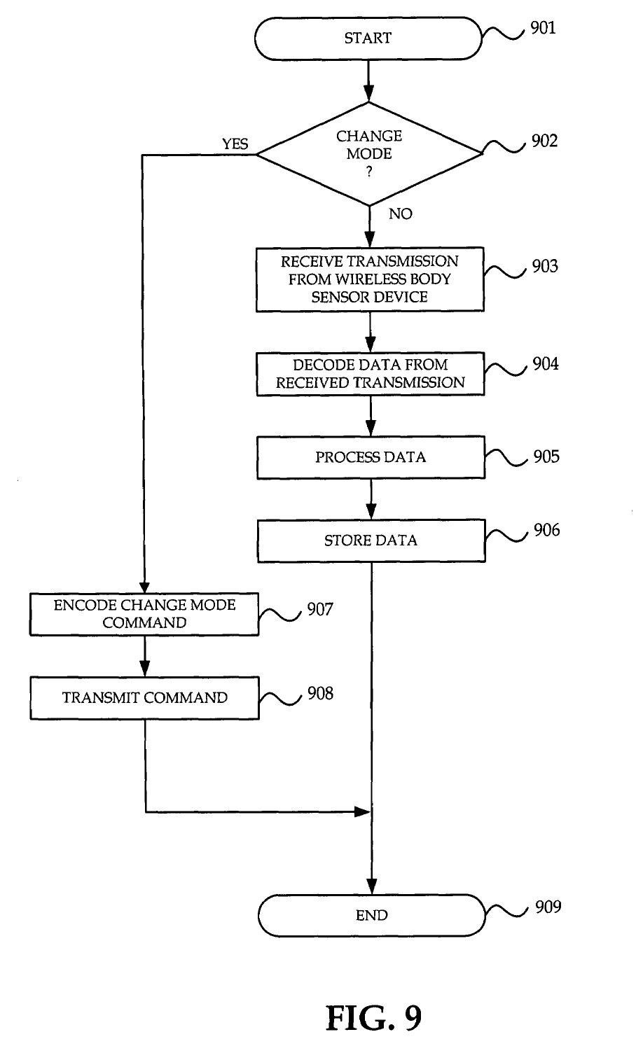

FIG. 9 is a flow diagram (900) illustrating an implementation of a method employed by a system for receiving and processing bio-potential data from a wireless body sensor device that is arranged in accordance with the present disclosure. Processing begins at block 901, and proceeds to decision block 902 where the computing system (e.g., 140, 210, 220, 230, 240, etc.) determines if the operating mode (e.g., relay mode or non-relay mode) for the wireless sensor devices needs to be changed. When the mode is to be changed processing continues to block 907 where the change mode command is encoded and transmitted to the wireless sensor device at block 908. When the mode is not changed, processing continues from decision block 902 to block 903 where a transmission is received from a wireless body sensor device. After the transmission is received, data is decoded from the transmission at block 904, processed at block 905 and stored at block 906. Processing terminates at block 909.

When measurements want to be made while the person is moving actively and perhaps move over many meters of travel, local connections to a personal computer is not feasible. In this case the radio system links the communication to a portable computing device such as the wrist watch or cell phone or PDA devices previously described. The data that is received by the portable computing device may be partially analyzed, and a few very simple statistics may be shown on the display of the portable computing device such as, for example the rate of a signal characteristic. However, the bio-potential data and/or statistics of the data are stored on the portable computing device so that they can be transferred to a network and/or to a personal computer for a more complete analysis of the bio-potential data.

Since there are common practices in viewing some bio-potentials such as EEGs, the data retrieved from the wireless body sensor devices can be post processed to show an estimate or approximate display of what the voltages would have been with common, long wired measurement techniques. This processing is facilitated by placing many wireless body sensor devices on the user and collecting bio-potential data from an array of such sensors. Each sensor can utilize four probes as illustrated in the figures, where the MEMS switch array in the sensor is configured to switch between six different measurements on an on-going basis during real-time processing. For example, assume the bio-potential sensors are numbered 1-4. Sensor measurements can be made across sensor pairs 1 and 2; 1 and 3; 1 and 4; 2 and 3; 2 and 4; and sensor pair 3 and 4. The overall sensory data can then be processed to get a more exact picture of the bio-potential information. In some instances the bio-potential information can be represented as a vector in three-dimensional space.

After the bio-potential data from all of the wireless body sensor devices is collected, a mapping of all of the sensors can be constructed so that an interpolation can be made between adjacent sensors. Moreover, estimation algorithms can be applied so that an accurate estimate of bio-potential information can be made based on the collected data.

The above specification, examples and data provide a complete description of the manufacture and use of the composition of the invention. Since many embodiments of the invention can be made without departing from the spirit and scope of the invention, the invention resides in the claims hereinafter appended.

I claim:

1. A wireless body sensor device for collecting bio-potential measurements from a user, wherein the wireless body sensor device has a small form factor that is defined according to an area associated with a watch battery, the wireless body sensor device comprising:
a first bio-potential probe that is in contact with the user at a first location, wherein the first bio-potential probe is arranged to provide a first input signal in response to the bio-potential measurements from the user at the first location;
a second bio-potential probe that is in contact with the user at a second location, wherein the second bio-potential probe is arranged to provide a second input signal in response to the bio-potential measurements from the user at the second location, wherein the distance between the first location and the second location is less than 2 cm; and
a wireless electronic system that has a form factor matched to the area associated with the watch battery, the wireless electronic system comprising:
a signal amplifier that is arranged to amplify a difference between the first input signal and the second input signal to provide an amplified signal;
an analog-to-digital converter circuit that is arranged to convert the amplified signal to a digital signal;
a digital signal processor that is arranged to encode the digital signal for transmission as an encoded signal;
a radio system that is arranged to generate a transmission signal from the encoded signal; and
an antenna system that is arranged in cooperation with the radio system to wirelessly transmit the transmission signal as a wireless transmission, wherein the wireless transmission is arranged for reception by either another wireless body sensor device or a computing device.

2. The wireless body sensor device of claim 1, further comprising:
a third bio-potential probe that is in contact with the user at a third location, wherein the third bio-potential probe is arranged to provide a third input signal in response to the bio-potential measurements from the user at the third location; and
a fourth bio-potential probe that is in contact with the user at a fourth location, wherein the fourth bio-potential probe is arranged to provide a fourth input signal in response to the bio-potential measurements from the user at the fourth location,
wherein the wireless electronic system further comprises a second signal amplifier that is arranged to amplify a difference between the third input signal and the fourth input signal to provide a second amplified signal; and
wherein the analog to digital converter circuit is also arranged to convert the second amplified signal to a second digital signal, wherein the digital signal processor is further arranged to process the second digital signal into the encoded signal.

3. The wireless body sensor device of claim 1, wherein the wireless electronic system further comprises a MEMS switch array that is arranged to selectively switch the first input signal and the second input signal to the inputs of the signal amplifier such that the inputs of the signal amplifiers are auto-zeroed.

4. The wireless body sensor device of claim 1, wherein the wireless electronic system further comprises a MEMS switch array that is arranged to selectively switch the first input signal and the second input signal to the inputs of the signal amplifier such that the input referred offset of the amplifier is removed.

5. The wireless body sensor device of claim 1, wherein the wireless electronic system further comprises a MEMS switch array that is arranged to selectively switch the first input signal and the second input signal to the inputs of the signal amplifier such that the signal amplifiers are chopper stabilized with a desired noise shaping characteristic.

6. The wireless body sensor device of claim 1, where the radio system and the antenna system in the wireless electronic system are arranged to cooperate with one another such that the wireless transmission corresponds to one of a Bluetooth transmission, a radio frequency (RF) transmission, and an ultra-wideband (UWB) transmission.

7. The wireless body sensor device of claim 1, where the antenna system includes a signal shaper that is coupled to an antenna, wherein the signal shaper is arranged to pre-distort the transmission signal to compensate for the signal dispersion characteristics of the antenna such that the signal transmitted by the antenna corresponds to a dispersion free Gaussian mono-pulse signal in the time domain.

8. The wireless body sensor device of claim 1, wherein the wireless electronics system further comprises a control processor that is arranged to control the operation of the radio system.

9. The wireless body sensor device of claim 8, wherein the control processor is arranged to periodically disable the radio system to conserve power.

10. The wireless body sensor device of claim 8, wherein the control processor is arranged to periodically configure the radio system to receive signals from the antenna.

11. The wireless body sensor device of claim 8, wherein the control processor is arranged to change to a relay mode when a change mode command is received by the radio system, wherein the control processor is arranged to configured the radio system such that the radio system receives signals from the antenna and retransmits the received signals during the relay mode, wherein the received signals correspond to bio-potential data from another wireless body sensor device.

12. The wireless body sensor device of claim 8, wherein the wireless electronics system is arranged to receive transmissions via the antenna system and the radio system when the relay mode is active, and wherein the radio system and the further comprises a control processor that is arranged to select a relay mode for the wireless body sensor device in response to a change mode command.

13. A method for collecting bio-potential measurements from a user with an array of wireless body sensor devices, wherein the wireless body sensor device has a small form factor that is defined according to an area associated with a watch battery, the method comprising:
receiving one of a plurality of wireless transmissions from a respective one of a plurality of wireless body sensor devices, wherein the received wireless transmission includes bio-potential data encoded therein;
decoding the bio-potential data from the received transmission;
identifying the received transmission with a corresponding Cartesian coordinate that is associated with a location of the corresponding wireless body sensor on the user; and
associating the bio-potential data with the identified Cartesian coordinate; and
storing the bio-potential data.

14. The method of claim 13, further comprising: remapping the bio-potential data from the Cartesian coordinate to another representation that is suitable for health care workers.

15. The method of claim 13, further comprising identifying two adjacent wireless body sensor devices from their respective Cartesian coordinates, interpolating between stored bio-potential data associated with the identified adjacent wireless body sensor devices.

16. System for wirelessly collecting bio-potential measurements from an array of locations associated with the user, the system comprising:
a plurality of wireless body sensor devices, wherein each wireless body sensor device is a small form factor device that is located at a corresponding one of the array of locations, wherein each wireless body sensor comprises:
a first bio-potential probe that is in arranged to provide a first input signal;
a second bio-potential probe that is in arranged to provide a second input signal;
a third bio-potential probe that is in arranged to provide a third input signal;
a fourth bio-potential probe that is arranged to provide a fourth input signal, wherein the first, second, third and fourth bio-potential probes are arranged radially about the corresponding location with a radius of less than 1 cm; and
a wireless electronic system that is centrally located with respect to the first, second, third, and fourth bio-potential probes, the wireless electronic system comprising:
a first signal amplifier with a first input terminal and a second input terminal, wherein the first signal amplifier is arranged to provide a first amplified signal in response to signals received at the first and second input terminals;
a second signal amplifier with a third input terminal and a fourth input terminal, wherein the second signal amplifier is arranged to provide a second amplified signal in response to signals received at the third and fourth input terminals;
a MEMS switch array that is arranged to selectively couple two of the first, second, third and fourth input signals to the first and second input terminals, and also arranged to selectively couple the other two of the first, second, third and fourth input signals to the third and fourth input terminals;
an analog-to-digital converter circuit that is arranged to convert the first amplified signal to a first digital signal and also convert the second amplified signal to a second digital signal;
a digital signal processor that is arranged to encode the first digital signal and the second digital signal for transmission as an encoded signal;
a radio system that is arranged to generate a transmission signal from the encoded signal; and
an antenna system that is arranged in cooperation with the radio system to wirelessly transmit the transmission signal as a wireless transmission.

17. The system of claim 16, further comprising: a computing device that is arranged to receive wireless transmissions, and retransmit the wireless transmissions when a relay mode is active for the computing device.

18. The system of claim 17, wherein the computing device comprises one of: a personal computer, a laptop computer, a cellular telephone, wrist watch, and a personal data assistant.

19. The system of claim 16, further comprising: a computing device that is arranged to create a mapping between each wireless body sensor device and coordinate representation that is suitable for health care workers.

20. The method of claim 16, wherein each wireless body sensor further comprises a control processor that is arranged to control the MEMS switch array such a plurality of bio-potential measurements are performed for each corresponding location by selectively changing the selection of the first, second, third, and fourth input terminals of the first and second signal amplifiers.
